(19) United States
(12) Patent Application Publication
(10) Pub. No.: US 2024/0252279 A1
(43) Pub. Date: Aug. 1, 2024

(54) DENTAL LAMINATE

(71) Applicant: e32 Technology Inc., Chantilly, VA (US)

(72) Inventors: Yong Min PARK, Chantilly, VA (US); Young Cheol CHANG, Chantilly, VA (US)

(73) Assignee: e32 Technology Inc., Chantilly, VA (US)

(21) Appl. No.: 18/102,543

(22) Filed: Jan. 27, 2023

(51) Int. Cl. A61C 5/30

(52) U.S. Cl. CPC A61C 5/30

(57) ABSTRACT

Provided is a dental laminate including a first structure having a curvature corresponding to at least a part of a first tooth and a shape corresponding to a front surface of the first tooth to cover the front surface of the first tooth, a second structure adjoining the first structure and having a curvature corresponding to at least a part of a second tooth adjoining the first tooth and a shape corresponding to a front surface of the second tooth to cover the front surface of the second tooth, and a bridge structure connecting the first structure to the second structure, wherein at least a part of the bridge structure is located between the first tooth and the second tooth.

### TECHNICAL FIELD

The present disclosure relates to a dental laminate, and more particularly, to an attachable and detachable dental laminate providing an aesthetic effect by covering the outer area of user's teeth.

### BACKGROUND ART

Originally, teeth are a body organ that not only performs the ingestion process including food intake, mastication, swallow, etc. and the function of vocalization of language but also is most exposed to others when we laugh. Because of this, even and regular teeth arrangement (dentition) and color are considered as an important factor that determines people's impressions, and accordingly, there has been a high demand for even and regular teeth and tooth whitening.

The orthodontic treatment takes a long time, and during the orthodontic treating, even when the mouth is closed, it protrudes due to the braces, which is not visually pleasing, and recently, it has been found that the orthodontic treatment itself may cause excessive stress to the skeletal structure.

In addition, the teeth whitening treatment which is performed in a dental clinic when teeth are discolored due to a cavity, teeth staining, etc. may have problems related to high cost and long time and may also have side effects such as smarting or discoloration of gums.

In case of broken or shortened teeth due to an accident or abrasion, dental plastic surgeries, implant treatments, resin treatments, etc. may be performed but there are still problems of cost and time.

Accordingly, among those who have uneven or discolored teeth, there has been a growing need for looking like having a beautiful smile with whiter or better colored and better arranged teeth quickly and easily for special occasions or events, such as photo-taking, meeting clients in person or online, attending a friend's wedding, at an important job interview, etc.

### DISCLOSURE

### Technical Goal

A technical goal according to an embodiment of the present disclosure is to provide a dental laminate that is easily placed on the surface of teeth temporarily to provide an effect of making a user look like having a beautiful smile with whiter or better colored and better arranged teeth.

Another technical goal according to an embodiment is to provide a dental laminate that is easily and stably placed and fixed on the outer surface of teeth and is easily attached to and detached from the outer surface of teeth according to a user's intention.

The technical goals to be achieved by the present disclosure are not limited to the forgoing, and other technical goals which are not mentioned above may be clearly understood by those skilled in the art from the description below.

### Technical Solution

To achieve the technical goals, provided is a dental laminate including a first structure having a curvature corresponding to at least a part of a first tooth and a shape corresponding to a front surface of the first tooth to cover the front surface of the first tooth, a second structure adjoining the first structure and having a curvature corresponding to at least a part of a second tooth adjoining the first tooth and a shape corresponding to a front surface of the second tooth to cover the front surface of the second tooth, and a bridge structure connecting the first structure to the second structure, wherein at least a part of the bridge structure is located between the first tooth and the second tooth.

In an embodiment of the present disclosure, the first structure may include a first surface and a second surface which face each other, the first surface may be arranged to face the front surface of the first tooth, and the first surface and the second surface may have different curvature surfaces from each other.

In an embodiment of the present disclosure, the first structure may include a first surface and a second surface which face each other, the first surface may be arranged to face the front surface of the first tooth, and at least a part of the first surface may have a curvature corresponding to a shape of the front surface of the first tooth.

In an embodiment of the present disclosure, the first structure may include a first extension extending toward an embrasure between the first tooth and the second tooth, the second structure may include a second extension extending toward the embrasure, and the bridge structure may be located between the first extension and the second extension.

In an embodiment of the present disclosure, at least a part of the bridge structure may be located in an embrasure between the first tooth and the second tooth.

In an embodiment of the present disclosure, the bridge structure may be longer than a depth of the embrasure.

In an embodiment of the present disclosure, the dental laminate may further include a protrusion extending between the first tooth and the second tooth from the bridge structure.

In an embodiment of the present disclosure, the protrusion may be provided to be inserted into an embrasure between the first tooth and the second tooth.

In an embodiment of the present disclosure, the protrusion may be shorter than a thickness of the first tooth or the second tooth.

In an embodiment of the present disclosure, the first structure may include a first surface and a second surface which face each other, the first surface may be arranged to face the front surface of the first tooth, and the first surface and the second surface may have different surface roughness from each other.

In an embodiment of the present disclosure, the first structure may include a first surface and a second surface which face each other, the first surface may be arranged to face the front surface of the first tooth, and the dental laminate may further include a third structure located between the first surface and the front surface of the first tooth.

In an embodiment of the present disclosure, the third structure may at least include a surface which faces the front surface of the first tooth and has different surface characteristics than the first surface.

In an embodiment of the present disclosure, the third structure may at least include an adhesive material.

In an embodiment of the present disclosure, the first structure may further include a first tip structure extending toward at least a part of an incisal surface of the first tooth.

In an embodiment of the present disclosure, a length of the first tip structure may be shorter than a thickness of the incisal surface.

### Advantageous Effects

According to an embodiment, provided is a dental laminate that is easily placed on a surface of a tooth to at least temporarily provide an effect of making a user look like having even and beautiful teeth and tooth color.

In addition, according to another embodiment, provided is a dental laminate that is easily and stably placed and fixed on the surface of teeth, and is easily attached to and detached from the surface of teeth according to a user's intention.

The effects of the present disclosure are not limited to the above effects, and should be understood as including all effects that can be inferred from the detailed description of the present disclosure or the features described in the claims of the present disclosure.

### DESCRIPTION OF DRAWINGS

FIG. 1A is a front view of a dental according to an embodiment, and FIG. 1B is a rear view of a dental laminate according to an embodiment.

FIGS. 2A and 2B are conceptual diagrams showing the difference before and after wearing a dental laminate according to an embodiment.

FIGS. 3A and 3B are conceptual views showing an area of a dental laminate according to an embodiment.

FIG. 4A is a cross-sectional view of a dental laminate according to an embodiment. FIGS. 4B and 4C are enlarged views of region A of FIG. 4A.

FIG. 5A is a cross-sectional view of a dental laminate according to an embodiment. FIGS. 5B and 5C are enlarged views of region B of FIG. 5A.

FIG. 6 is a conceptual view showing a dental laminate according to an embodiment.

### MODE FOR INVENTION

Hereinafter, the present disclosure will be described with reference to the accompanying drawings. However, the present disclosure may be implemented in various different forms and is not limited to embodiments of the present disclosure described herein. To clearly describe the present disclosure, parts that are not associated with the description have been omitted in the drawings, and throughout the specification, like reference numerals denote like parts.

While such terms as “first,” “second,” “A,” “B,” etc., may be used to describe various components, such components must not be limited by the above terms. The above terms are used only to distinguish one component from another. For example, without departing from the scope of rights of the present disclosure, a first component may be named as a second component, and similarly, the second component may be named as the first component. The term “and/or” includes any combination of a plurality of related recited items or any of a plurality of related recited items.

Throughout the specification, when a portion is described as being connected to (coupled to, in contact with, or combined with) another portion, the portion may be connected directly to another portion, or indirectly connected to another portion with an intervening portion therebetween. When a portion “includes” a component, another component may be further included, rather than excluding the existence of the other component, unless otherwise described.

The terms used in the specification are merely used to describe exemplary embodiments, and are not intended to limit the present disclosure. An expression used in the singular encompasses the expression of the plural, unless it has a clearly different meaning in the context.

Further, the terms such as “include” or “have” in various embodiments of the present disclosure are used to specify the existence of features, numbers, processes, operations, components, parts recited in the detailed description, or combinations thereof, and thus should not be understood as pre-excluding the existence or possibility for addition of one or more other features, numbers, processes, operations, components, parts, or combinations thereof.

When a component is described as being “on” or “above” another component, the component may be directly on another component or indirectly on another component with an intervening component therebetween.

As described above, among those who have uneven or discolored teeth, there has been a growing need for looking like having a beautiful smile with whiter or better colored and better arranged teeth quickly and easily for special occasions or events, such as photo-taking, meeting clients in person or online, attending a friend's wedding, at an important job interview, etc., without a time-consuming treatment, such as orthodontic treatment, etc. Thus, a dental laminate that is easily placed on the surface of teeth temporarily to provide an effect of making a user look like having a beautiful smile with whiter or better colored and better arranged teeth is required.

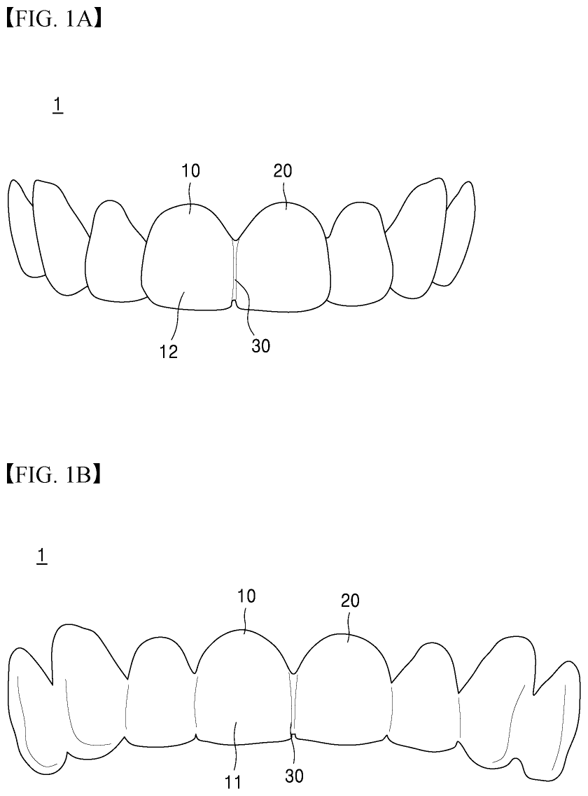

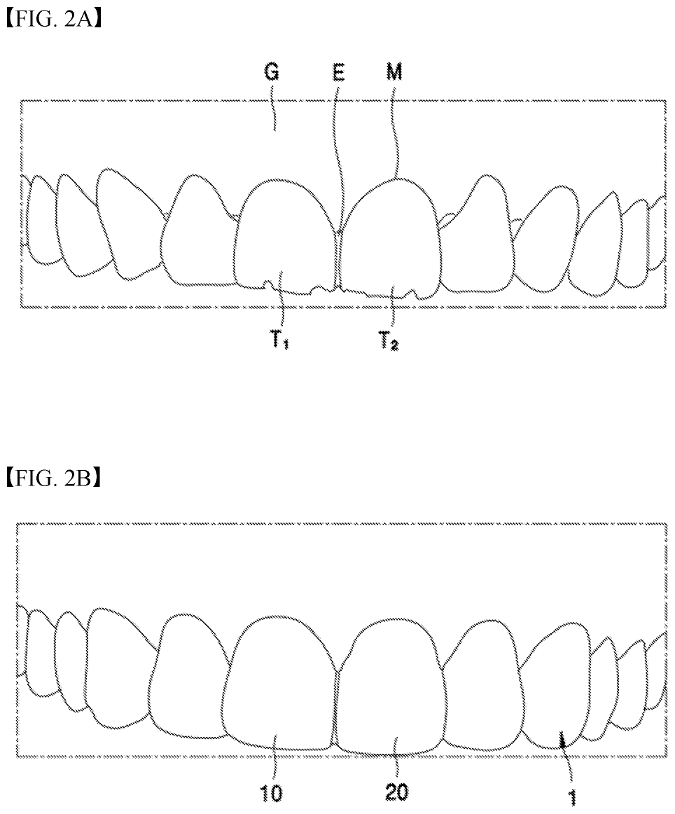

FIG. 1A is a front view of a dental according to an embodiment, and FIG. 1B is a rear view of a dental laminate according to an embodiment. FIGS. 2A and 2B are conceptual diagrams showing the difference before and after wearing a dental laminate according to an embodiment.

With reference to FIGS. 1A, 2A, and 2B, to achieve the aforementioned technical goals, an embodiment may include a plurality of structures respectively corresponding to front surfaces of at least two adjoining teeth and at least one bridge structure connecting the structures to each other. Optionally, in an embodiment, provided is a dental laminate 1 including a first structure 10 having a shape corresponding to a front surface of a first tooth T1, a second structure 20 adjoining the first structure 10 and having a shape corresponding to a front surface of a second tooth T2, and a bridge structure 30 connecting the first and second structures 10 and 20 to each other.

The first structure 10 may have a curvature corresponding to at least a part of the first tooth T1 and may cover at least a part of the front surface (labial-side region) of the first tooth T1. At least a part of the bridge structure 30 may be positioned between the first tooth T1 and the second tooth T2.

According to such a structure, a user who has uneven teeth or a broken tooth as shown in FIG. 2A may look like having even and beautiful teeth and tooth color as shown in FIG. 2B, by placing the dental laminate 1 on the teeth. At the same time, it may be possible to maximize visual aesthetic effects by minimizing a sense of difference between adjacent teeth and to easily attach and detach the dental laminate.

In an embodiment, the first structure 10 and the second structure 20 may each include a material which has the same or similar color with the color of teeth and remains stable against external impact, for example, a material used for oral appliances such as dentures and braces. For example, poly methyl methacrylate (PMMA), poly 2-methoxyethyl acrylate (PMEA), etc. may be used, and in an embodiment, Dentsply Sirona's Lucitone may also be used, but the present disclosure is not limited to thereto. Any materials generally used for oral appliances such as dentures, braces, etc. should be construed as being included in the scope of the present disclosure.

In an embodiment, the first structure 10 and the second structure 20 may be manufactured using three-dimensional (3D) printing. As such, materials used for manufacturing oral appliances such as dentures, braces, etc. by using 3D printing, for example, Lucitone of Dentsply Sirona may be used as a material used in the manufacture of the first structure 10 and the second structure 20, but the present disclosure is not limited thereto. Any materials generally used for manufacture of oral appliances such as dentures, braces, etc. should be construed as being included in the scope of the present disclosure.

Hereinafter, although embodiments are described by taking the first structure 10 as an example for convenience of explanation, the structure and function of the first structure 10 may also be applied to the second structure 20 unless specified otherwise.

Referring back to FIGS. 1A and 1B, in an embodiment, the first structure 10 may include a first surface 11 and a second surface 12 which face each other, and the first surface 11 may be arranged to face the front surface of the first tooth T1. As the first structure 10 has such structure, the first surface 11 may be positioned to face the front surface of the first tooth T1, and thus, may be directly attached or adhered to the tooth.

Optionally, in an embodiment, the first surface 11 and the second surface 12 may have different curvature surfaces from each other. The first surface 11 may be arranged to face the front surface of the tooth and facilitate stable attachment or adherence of the dental laminate 1 to the tooth.

The second surface 12 may be a side that other people would see when a user wears the dental laminate 1.

The curvature surfaces of the first surface 11 and the second surface 12 are not particularly limited. The first surface 11 may vary depending on the size, curvature, etc. of the user's teeth, and the second surface 12 may vary depending on the curvature surface of the first surface 11, size and thickness of the tooth, etc.

The curvature surfaces of the first surface 11 and the second surface 12 may be measured or calculated using photos, video files, X-RAY pictures, etc. of the user's teeth. The method of measuring and calculating the curvature surfaces is also not particularly limited, and all methods easily selected by a person skilled in the art should be interpreted as being included in the scope of the present disclosure.

In an embodiment, at least a part of the first surface 11 may have a curvature corresponding to a shape of the front surface of the first tooth T1. Teeth are generally hard and each tooth has a certain curvature. As at least a part of the first surface 11 of the dental laminate 1 has a curvature corresponding to a shape of the front surface of the first tooth T1, the dental laminate 1 may be more stably attached or adhered to the teeth and may improve the adhesion to the teeth. According to an embodiment, the first surface 11 may have a 3D shape corresponding to the overall curvature of the front surface of the tooth. However, the present disclosure is not necessarily limited thereto, and the first surface 11 may have a curvature corresponding to at least a part of the front surface of the tooth to improve the adhesion in at least that part, compared to in other parts.

The second surface 12 may not necessarily have a curvature corresponding to the front surface of the tooth, and may optionally have an arbitrary curvature that looks most natural in appearance. The curvature of the second surface 12 may be determined by considering user's facial conditions, such as, size of the user's mouth, shape of the face, proportion of the mouth in the face, size of the face, etc. and/or user's needs. Optionally, the curvature of the second surface 12 may be selected and determined among most beautiful curvatures considering the gender of the user. Optionally, the curvature of the second surface 12 may be determined by conducting simulation in relation to the user's face.

In another embodiment, an overall and approximate curvature of the second surface 12 may be less than an overall and approximate curvature of the first surface 11. Accordingly, the first structure 10 may have a tension in a direction in which the tooth is covered, and such tension may further improve the adhesion when the first structure 10 is mounted on the tooth, which leads to more stable fixation.

In an embodiment, the first structure 10 may include the first surface 11 and the second surface 12 which have different surface roughness from each other. The first surface 11 may refer to a surface facing the front surface of the first tooth T1, and the second surface 12 may refer to the opposite surface of the first surface 11. As such, the first structure 10 may have a double-sided structure including one side attached to the tooth and the opposite side shown to others.

The first surface 11 may have a rough surface such that the dental laminate 1 is easily attached to the first tooth T1, and the second surface 12 may have a smooth surface having a lower roughness such that the user looks like having smooth teeth. When the surface roughness of the first surface 11 is increased, the adhesive force between the first surface 11 and the first tooth T1 may be improved by increasing a contact area of a medium arranged between the first tooth T1 and the first surface 11. The medium may be in the form of moisture that is naturally present in a user, such as saliva or may be an adhesive material described in other embodiments below or a film-type medium.

The degree of roughness is not particularly limited, and the roughness of the first surface 11 may vary depending on the roughness of the tooth surface of the user wearing the dental laminate 1 or the size of the tooth, and the roughness of the second surface 12 may also vary depending on the roughness of the tooth surface of the user who wears the dental laminate 1 and the place or purpose of use.

According to an embodiment, the first structure 10 may be formed to have a larger area than the first tooth T1. Accordingly, the first structure 10 may completely cover the first tooth T1. Optionally, the first structure 10 may extend to an interdental embrasure between the first tooth T1 and the second tooth T2. Accordingly, even when a user has a large interdental embrasure, the embrasure may not be exposed to the outside. Of course, this can be equally applied to the adjacent second structure 20.

Optionally, according to another embodiment, the distance between the first surface 11 and the second surface 12 of the first structure 10, that is, the thickness of the first structure 10 may be formed to have a certain degree of rigidity and tension in the longitudinal direction and the first structure 10 may be formed to have a thickness that does not make the user look like having protruding teeth when the user wears the dental laminate.

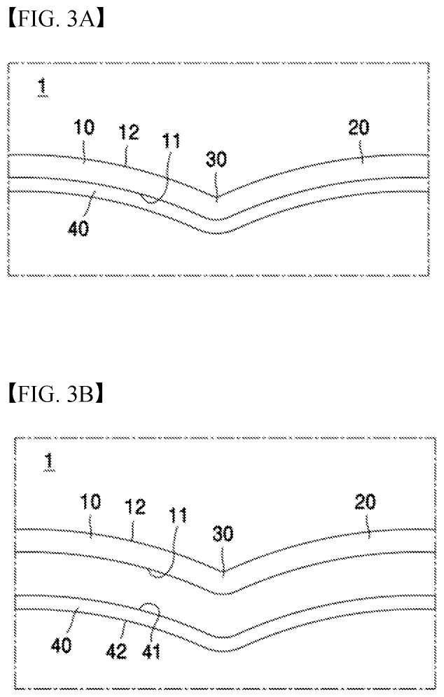

FIGS. 3A and 3B are conceptual views showing an area of a dental laminate according to an embodiment.

With reference to FIG. 3A, in an embodiment, the dental laminate 1 may further include a third structure 40 arranged adjacent to the first surface 11 and assisting in fixing or attaching of attaching the first surface 11 to the first tooth T1.

With reference to FIG. 3B, the third structure 40 may include a I surface 41 arranged between the third structure 40 and the first surface 11 and a II surface 42 arranged opposite to the I surface 41.

The II surface 42 surface may face the front surface of the first tooth T1 and may have different surface characteristics from the first surface 11 of the first structure 10. For example, the II surface 42 may have a surface characteristic of greater attachment force to the first tooth T1, compared to the first surface 11.

The surface characteristics may refer to characteristics that determine the attractive force between two adjacent objects, such as attachment, combination, bonding, adsorption, adhesion, etc. between objects and include, for example, a degree of hydrophilicity/hydrophobicity, a roughness, etc.; however, the present disclosure is not limited thereto, and any surface characteristics controlled for attachment, combination, bonding, adsorption, or adhesion between objects that can be easily selected by a person skilled in the art should be construed as being included in the scope of the present disclosure.

In an embodiment, the third structure 40 may at least include an adhesive material. The adhesive material may refer to a material used to temporarily but more stably fix the dental laminate 1 to the front surface area of the tooth, for example, a dental adhesive cream. However, the present disclosure is not limited thereto, and any attaching materials, combinative materials, bonding materials, adsorptive materials, or adhesive materials that can be used to temporarily fix dentures, artificial teeth, teeth, dental instruments, etc., and can be easily selected by a person skilled in the art should be construed as being included in the scope of the present disclosure.

As such, the adhesive force between the structures and the teeth may be further increased by the third structure 40.

Referring back to FIG. 2A, a gingival margin M may be formed between the first tooth T1 and the gum, and an embrasure E may be formed among the first tooth T1, the second tooth T2 and the gum G. The gingival margin M and the embrasure E may vary depending on the size of the teeth, the size difference between the first tooth T1 and the second tooth T2, the condition of the gum, the plague stage, etc., and each person may have different position, size, and shape of the gingival margin M and the embrasure E.

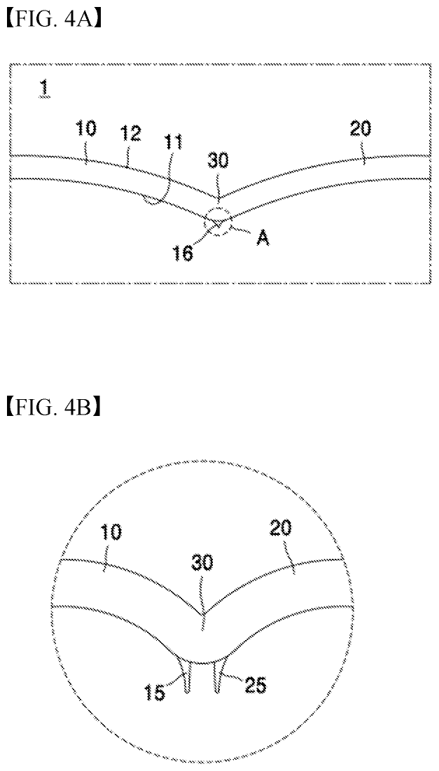

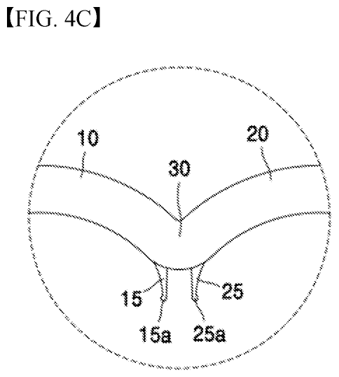

FIG. 4A is a cross-sectional view of a dental laminate according to an embodiment. FIGS. 4B and 4C are enlarged views of region A of FIG. 4A.

With reference to FIG. 4A, in an embodiment, the first structure 10 and the second structure 20 may include at least one extension 16 extending towards the embrasure E between the first tooth T1 and the second tooth T2. The embrasure E may be formed among the first tooth T1, the second tooth T2, and the gum G, and by arranging the extension 16 in the embrasure E, the dental laminate 1 may be more stably arranged and fixed onto the front surface area of the teeth.

With reference to FIG. 4B, in an embodiment, the first structure 10 may include a first extension 15 extending toward the embrasure E between the first tooth T1 and the second tooth T2. As the first structure 10 has such structure, when the dental laminate 1 is worn, the first extension 15 may be arranged in the embrasure E, and accordingly, the dental laminate 1 may be more stably placed on the front surface area of teeth.

With reference to FIG. 4B, in another embodiment, the second structure 20 may include a second extension 25 extending toward the embrasure E. According to such structure, the first extension 15 and the second extension 25 respectively extending from the first structure 10 and the second structure 20 which are adjacent to one embrasure E may be provided to stably fix the dental laminate 1 over the areas of the front surface of the teeth.

With reference to FIG. 4C, in an embodiment, the extension 16 may have a structure that easily passes through a hole during an insertion process, but does not easily get out of the hole in a detachment process. For example, the extension 16 may include an end portion (15a and 25a) having a hook shape or a J-shape. As the extension 16 has such structure, the dental laminate 1 may be more stably fixed to the front surface area of the teeth. However, the present disclosure is not limited thereto, and it may be sufficient to form the end portion of the extension 16 to be slightly thicker than other parts to provide a certain degree of fixation power when the dental laminate is worn. Any other method of fixing objects by using protrusions should be construed as being included in the scope of the present disclosure.

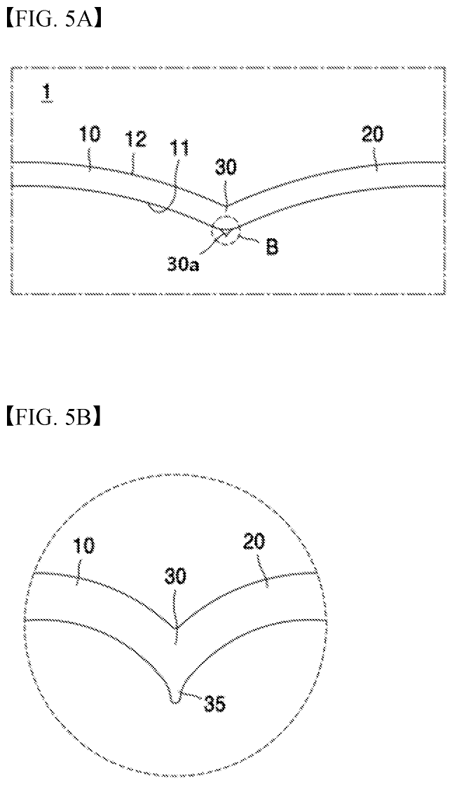

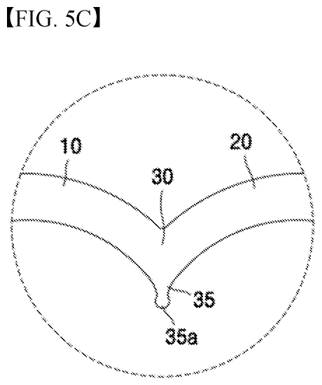

FIG. 5A is a cross-sectional view of a dental laminate according to another embodiment. FIGS. 5B and 5C are enlarged views of region B of FIG. 5A.

With reference to FIG. 5A, in an embodiment, the dental laminate 1 may include a protruding area 30a protruding from the bridge structure 30. As such, as at least a part of the bridge structure 30 is positioned in the embrasure E between the first tooth T1 and the second tooth T2, at least a part of the bridge structure 30 may be inserted into the embrasure E, and accordingly, the dental laminate 1 may be more stably fixed to the front surface area of the teeth.

In an embodiment, the bridge structure 30 may be longer than or equal to the depth of the embrasure E. As the bridge structure 30 including the protruding area 30a is arranged in the embrasure E, the dental laminate 1 may be stably fixed to the front surface area of the teeth.

To more stably fix the dental laminate 1 to the front surface area of the teeth, the bridge structure 30 may be longer than the depth of the embrasure E such that the bridge structure 30 is inserted deeply in the depth direction of the embrasure E. Through this, the dental laminate 1 may be more stably fixed or attached to the front surface area of the teeth.

With reference to FIG. 5B, in an embodiment, the dental laminate 1 may further include a protrusion 35 extending between the first tooth T1 and the second tooth T2 from the bridge structure 30. The protrusion 35 may be formed to be directed towards a contact surface, the embrasure E, etc. between the first tooth T1 and the second tooth T2; however, to more stably fix the dental laminate 1 to the front surface area of the teeth, the protrusion 35 may be formed to be directed toward the embrasure E between the first tooth T1 and the second tooth T2.

In an embodiment, the protrusion 35 may be formed to be inserted into the embrasure E formed between the first tooth T1 and the second tooth T2. By having such structure, the protrusion 35 may be combined with and inside the embrasure E, and the dental laminate 1 may be more stably fixed to the front surface area of the teeth.

With reference to FIG. 5C, in an embodiment, the end portion 35a of the protrusion 35 may have a wider cross-sectional area than at least some of other areas of the protrusion 35. By having such structure, when the protrusion 35 is inserted into the embrasure E formed between the first tooth T1 and the second tooth T2, the end portion 35a of the protrusion 35 may be more stably combined between the first tooth T1 and the second tooth T2 such that the dental laminate 1 is more stably attached or fixed to the teeth.

Although it is not shown in the drawings, in an embodiment, the protrusion 35 may be formed to protrude toward a contact surface between the first tooth T1 and the second tooth T2. According to such structure, even when a user with weak gums due to periodontitis, etc. repetitively attaches/detaches the dental laminate 1, no damage may be caused to the teeth.

In an embodiment, the protrusion 35 may be shorter than the thickness of the first tooth T1 or the second tooth T2. When the protrusion 35 is formed to protrude toward the contact surface between the first tooth T1 and the second tooth T2, as the protrusion 35 is shorter than the thickness of the first tooth T1 and the second tooth T2, the protrusion 35 may not pass through the contact surface between the first tooth T1 and the second tooth T2, and thus, no damage may be caused to the teeth of a user with weak teeth.

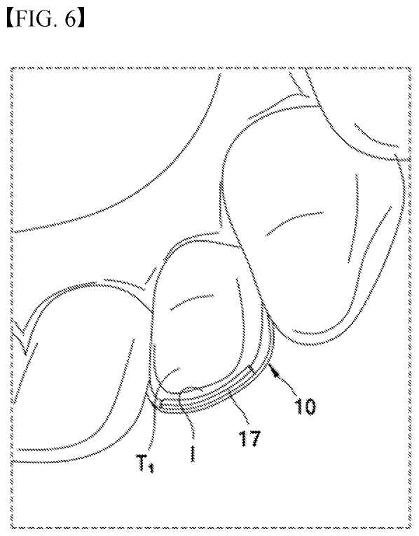

FIG. 6 is a conceptual view showing a dental laminate according to another embodiment.

With reference to FIG. 6, in an embodiment, the first structure 10 may further include a first tip structure 17 extending toward at least a part of an incisal surface I of the first tooth T1. The incisal surface I may refer to an end surface of a tooth that is used when cutting the food or an object to be chewed.

According to such structure, when the user wears the dental laminate 1, the first tip structure 17 may be arranged on at least a part of the incisal surface I of the first tooth T1 and the dental laminate 1 may be held and fixed by the end portion of the tooth. In this manner, the dental laminate 1 may be more stably fixed to the tooth.

The first tip structure 17 may vary according to the size and shape of the tooth, and more specifically, when the thickness of the tooth is great, the first tip structure 17 may be as large as the thickness of the tooth.

In an embodiment, the length of the first tip structure 17 may be shorter than the thickness of the incisal surface I. As the first tip structure 17 has a shape that covers only an area of the incisal surface I of the tooth, the dental laminate 1 may be more stably attached to the tooth and also the dental laminate 1 may be easily detached from the tooth.

In an embodiment, the length of the first tip structure 17 may be greater than the thickness of the incisal surface I. As the first tip structure 17 completely covers the incisal surface I and thus slightly envelops the end surface of the tooth, the dental laminate 1 may be more stably attached to the tooth.

The first tip structure 17 may have flexibility such that the first tip structure 17 is stably combined with the end surface of the tooth. To this end, the first tip structure 17 may be thinner than other portions of the first structure. Optionally, the first tip structure 17 may further include a flexible material. Such flexible material is not particularly limited, and any flexible materials that a person skilled in the art may easily select from materials harmless to humans for use in oral appliances or dental instruments should be construed as being included in the scope of the present disclosure.

Of course, the embodiments described above may be combined with each other for application.

The above descriptions of the present disclosure are provided merely as an example, and a person skilled in the art to which the present disclosure pertains may understand that the embodiments can be easily modified into other specific forms without changing technical ideas or essential technical features of the present disclosure. Therefore, it should be understood that the embodiments described above are provided as an example in every aspects, and thus do not pose a limitation on the present disclosure. For example, each component described as a single type component may be implemented in a dispersed manner, and similarly to this, components described as being dispersed may be implemented in an integrated manner.

The scope of the present disclosure is defined by the appended claims, and should be construed as encompassing all modifications or adaptions derived from the meaning and scope of the claims and their equivalent concepts.

### INDUSTRIAL APPLICABILITY

Provided is a dental laminate that is easily placed on the surface of teeth temporarily to provide an effect of making a user look like having a beautiful smile with whiter or better colored and better arranged teeth.

1. A dental laminate comprising:
a first structure having a curvature corresponding to at least a part of a first tooth and a shape corresponding to a front surface of the first tooth to cover the front surface of the first tooth;
a second structure adjoining the first structure and having a curvature corresponding to at least a part of a second tooth adjoining the first tooth and a shape corresponding to a front surface of the second tooth to cover the front surface of the second tooth; and
a bridge structure connecting the first structure to the second structure,
wherein at least a part of the bridge structure is located between the first tooth and the second tooth.

2. The dental laminate of claim 1, wherein the first structure includes a first surface and a second surface which face each other,
the first surface is arranged to face the front surface of the first tooth, and
the first surface and the second surface have different curvature surfaces from each other.

3. The dental laminate of claim 1, wherein the first structure includes a first surface and a second surface which faces each other,
the first surface is arranged to face the front surface of the first tooth, and
at least a part of the first surface has a curvature corresponding to a shape of the front surface of the first tooth.

4. The dental laminate of claim 1, wherein the first structure includes a first extension extending toward an embrasure between the first tooth and the second tooth,
the second structure includes a second extension extending toward the embrasure, and
the bridge structure is located between the first extension and the second extension.

5. The dental laminate of claim 1, wherein at least a part of the bridge structure is located in an embrasure between the first tooth and the second tooth.

6. The dental laminate of claim 5, wherein the bridge structure is longer than a depth of the embrasure.

7. The dental laminate of any claim 1,
further comprising a protrusion extending between the first tooth and the second tooth from the bridge structure.

8. The dental laminate of claim 7, wherein the protrusion is provided to be inserted into the embrasure between the first tooth and the second tooth.

9. The dental laminate of claim 7, wherein the protrusion is shorter than a thickness of the first tooth or the second tooth.

10. The dental laminate of claim 1, wherein the first structure includes a first surface and a second surface which face each other,
the first surface is arranged to face the front surface of the first tooth, and
the first surface and the second surface have different surface roughness from each other.

11. The dental laminate of claim 1, wherein the first structure includes a first surface and a second surface which face each other,
the first surface is arranged to face the front surface of the first tooth, and
the dental laminate further includes a third structure located between the first surface and the front surface of the first tooth.

12. The dental laminate of claim 11, wherein the third structure at least includes a surface which faces the front surface of the first tooth and has different surface characteristics than the first surface.

13. The dental laminate of claim 11, wherein the third structure at least includes an adhesive material.

14. The dental laminate of claim 1, wherein the first structure further includes a first tip structure extending toward at least a part of an incisal surface of the first tooth.

15. The dental laminate of claim 14, wherein a length of the first tip structure is shorter than a thickness of the incisal surface.

16. The dental laminate of claim 14, wherein a length of the first tip structure is greater than a thickness of the incisal surface.
